(19) United States
(12) Patent Application Publication
(10) Pub. No.: US 2001/0014316 A1
(43) Pub. Date: Aug. 16, 2001

(54) Liquid cleansing composition

(76) Inventor: Marie Harbeck, Austin, TX (US)

Correspondence Address: MARIE HARBECK, 3202 CLUMPGRASS COVE, AUSTIN, TX, 78735, US

(21) Appl. No.: 09/753,896

(22) Filed: Jan. 3, 2001

Related U.S. Application Data
(63) Continuation-in-part of application No. 09/248,573, filed on Feb. 11, 1999, now Pat. No. 6,193,987.

(51) Int. Cl. C11D 3/50; A61K 7/075; A61K 7/08; C11D 9/44; A61K 6/00; A61K 7/00

(52) U.S. Cl. 424/70.22; 510/101

(57) ABSTRACT

An improved and useful organic oil-based liquid cleansing composition for the topical transdermal delivery of hydrating and nourishing skin additives while gently cleansing the dermis including the alleviation and treatment of dermatitis, eczema, and lupus skin infirmities, as well as dry, sensitive, chapped, flaking and itching skin conditions, which has as its constituents, safflower oil, flaxseed oil, and tincture of benzoin in a glycerin base.

This is a “Continuation-In-Part” application for Ser. No. 09/248,573 filed Feb. 11, 1999 for Marie Harbeck.

### BACKGROUND

Field of Invention

This invention relates to an improved and useful organic oil-based liquid cleansing composition for the alleviation and treatment of dermatitis, eczema, and lupus skin infirmities, including dry, sensitive, chapped, flaking and itching skin conditions, which has as its main constituents, safflower oil, flaxseed oil, and tincture of benzoin in a glycerin base.

To the best of the applicant's knowledge the following is the most relevant prior art:

U.S. Pat. No. 4,740,367 Force, et al. 1988

U.S. Pat. No. 5,244,679 Freston. 1993

U.S. Pat. No. 5,489,429 Griat, et al. 1996

U.S. Pat. No. 5,683,704 Ohba, et al. 1997

U.S. Pat. No. 4,883,659 Goodman, et al. 1989

U.S. Pat. No. 4,608,392 Jacquet et al. 1986

U.S. Pat. No. 5,431,911 Reynolds. 1995

U.S. Pat. No. 6,147,040 Van der Hagen. 2000

U.S. Pat. No. 6,143,704 Van Gunst et al. 2000

U.S. Pat. No. 6,046,145 Santora et al. 2000

U.S. Pat. No. 5,683,683 Scafidi 1997

U.S. Pat. No. 6,107,262 Nobles, II. 2000

The cosmetic and pharmaceutical industry is continually expanding its efforts in order to provide topical soap preparations which will moisten and soften the skin, eliminate dryness, cracking, chapping, chaffing, redness and restore the skin to its natural healthy condition.

Synthetic surfactants, silicones, ammonia, alcohol's, solvents, acids, salts, artificial colors, fragrances, and the like are being incorporated into the compositions of the cited prior art to extend shelf life of the cited compositions that are supplied to stores and supermarkets.

Some of these components aggravate skin infirmities, thus the combinations of skin aggravating synthetic compositions employed continue to increase yearly.

It will be appreciated that the expansion and growth of the cited prior art has devolved into highly complex synthetic adjuvants for use in the formulations.

The problem with the use of the compositions of the cited prior art which make up the bulk of transdermal delivery systems are their chemical structures which are known to strip the acid mantle from the skin and contribute to hand, foot, scalp, eye and skin irritations.

Alcohol, acids, solvents, some synthetic perfumes, colorants, salts, and ammonia adjuvants incorporated into the compositions of the prior art cause dryness of the skin, rashes, allergies, dermatitis, peeling, bleeding, itching, painful cracking, dehydration and flaking of the skin.

Unfortunately, the cited prior art compositions are relatively ineffective for distressed skin conditions, not allowing the treatment and alleviation of skin irritations related to allergic skin reactions to synthetic adjuvants, so there is a need in the art for organic compounds for improved compositions for skin care.

The said prior art compositions do not employ the massive quantities of cold-pressed organic vegetable oils as does the present invention.

The said prior art compositions do not employ potent organic compositions that this invention incorporates due to prohibitive manufacturing costs.

In terms of effectiveness, the said cited prior art compositions do not provide satisfactory skin penetration and supplying properties that leave skin satiny smooth and rehydrated.

The said cited prior art compositions do not provide the high volume of cold pressed organic safflower oil, or flaxseed oil, in their formulas to immediately restore polyunsaturated oil lipids that are essential to nourish and maintain healthy skin.

The said cited prior art compositions do not provide the high volume of tocopheral linoleate compositions to their compositions.

The said cited prior art compositions do not include tincture of benzoin as an antiseptic adjuvant.

The said cited prior art compositions do not boil tincture of benzoin to eliminate its alcohol content.

The said cited prior art compositions do not provide an urgent need that exists for compositions like the present invention to the dermatological, pharmaceutical, alternative therapy and cosmetology fields.

The said cited prior art compositions do not provide organic compositions to alleviate and treat the symptoms of dermatitis, eczema, lupus and rosacea skin infirmities.

The said cited prior art compositions do not provide high quantities of organic essential oil of lavender to fight fungal infirmities.

The said cited prior art compositions do not provide organic polyunsaturated compounds that deposit oil lipids deep within the dermis while gently cleansing the skin during showering, washing or bathing.

The said cited prior art compositions do not provide organic moisturizing compositions that provide a protective water resistant, non-greasy, antiseptic and anti-fungal film on the skin.

The said cited prior art compositions do not provide organic compositions that are water resistant to body fluids, sebaceous gland activity and perspiration.

The said cited prior art compositions do not provide organic compositions to provide a high volume of organic essential oils as anti-itch additives.

The said cited prior art compositions do not provide a liquid soap composition that can be used as a skin cleanser and moisturizer for the skin and hair.

The said cited prior art compositions do not provide a high volume of organic safflower and flaxseed oils in the said compositions.

The said cited prior art compositions do not provide a high volume of organic compositions to replace lost oil lipids to the dermis that are stripped by the solvent action of mineral based additives.

The said cited prior art compositions do not provide gentle, organic compositions for infant and adolescent use.

Objectives and advantages of the present invention are:

a) to provide an alcohol free topical skin preparation,

b) to provide an essential oil as an organic antiseptic to treat bacterial, fungal and environmental aggressors of the skin,

c) to provide topical skin composition that has potent effectiveness in the treatment of skin infirmities, such as dermatitis, eczema, lupus, rosacea, chapping, rough skin, dryness, itching, cracking, flaking and skin allergies related to synthetic substances and the like;

d) to provide a high volume of organic cold pressed vegetable oil composition for immediate transdermal delivery,

e) to provide a satisfactory skin penetration of the said composition and supplying properties that leave the skin satiny smooth and rehydrated,

f) to provide a high volume of organic essential oils for anti-bacterial qualities and to perfume the said composition,

g) to provide a high volume of organic safflower and flaxseed oils in the said composition to replace lost oil lipids to the dermis,

h) to provide an urgent need that exists for composition like the present invention to the dermatological, pharmaceutical, alternative therapy and cosmetology fields,

i) to alleviate and treat the symptoms of dermatitis, rough skin, dryness, cracking, eczema, chapping, itching and allergic skin reactions due to ammonia, solvents, acids, alcohol substances, and synthetic perfumes and colorants;

j) to provide deep moisturizing composition while gently cleansing the skin during bathing or showering,

k) to provide gentle, organic composition for infant and adolescent use,

l) to provide an organic water resistant, non-greasy, antiseptic and moisturizing film on the skin,

m) to provide organic composition that is water resistant to body fluids, sebaceous gland activity and perspiration,

n) to provide organic composition without chemical structures that are known to cause skin irritations,

o) to provide organic composition without solvents, mineral oil, artificial colorants and ammonia additives; and

p) to provide an organic composition suitable for infant and adolescent use.

An urgent need exists for a skin composition like the present invention, that employs a high volume of potent cold-pressed organic flaxseed and safflower oils plus organic vitamins to gently cleanse and feed the skin with nutritive polyunsaturated oil lipids without the addition of skin irritating synthetic adjuvants to achieve its purposes. Further advantages of this invention are the employment of an organic essential plant oil that protects against fungal infirmities and fight environmental skin aggressors, leaving the skin in a healthy, hydrated, moist condition. Still further objects and advantages will become apparent from a consideration of the ensuing descriptions.

### BRIEF SUMMARY OF THE INVENTION

The improved invention provides a potent, swift acting, long lasting, non-sticky, organic, transdermal cleansing formulation for treating and alleviating dry, distressed skin conditions, including dermatitis and eczema, lupus, rosacea, rough skin, cracking, itching, peeling, redness, allergic skin reactions to alcohol and solvent sensitivity;

provides a liquid soap formulation which soothes, moisturizers and restores essential nutrients to the skin to improve its texture and protect it against dryness;

provides an organic cleansing formulation for the treatment and alleviation of itching, and redness of the skin;

provides a liquid soap formulation for the treatment and alleviation of dry, chapped, cracked, flaky, itching and allergy prone skin;

provides an organic formulation for moisturizing and protecting delicate infant and adolescent skin; and

provides an organic formulation for the treatment, alleviation and maintenance of cracked, callused and rough, skin.

These and other objects are accomplished in accordance with a particular embodiment of the present improved invention which provides a skin cleansing composition in the form of a liquid soap, comprising a mixture consisting of tincture of benzoin, tocopheral linoleate, organic cold-pressed flaxseed, safflower, jojoba, sweet almond, and apricot kernel oils, uncolored liquid glycerin soap, essential plant perfume, and mixtures thereof. The essential components of the present composition readily intermix to form a mixture suitable for topical cleansing and hydrating the skin. The present components are combined in such relative proportions so as to bring about a marked calming and moisturizing effect to the skin by supplying to the skin nutrients that are essential for healthy skin.

### DESCRIPTION OF THE INVENTION

An improved and useful liquid oil-based soap composition used to cleanse, and restore the skin to a healthy condition through the trandermal delivery of organic nutrients and vitamins in a glycerin base.

The present improved invention includes the surprising discovery that cold-pressed organic safflower and flaxseed oils, tincture of benzoin and high volume of tocopheral linoleate, can be used as active non-greasy principals in an improved skin care composition to be employed in skin cleansing, restoring and delivering nutrients that are essential for the maintenance of healthy skin, as well as alleviating and treating conditions attributed to lupus, rosacea, and eczema skin conditions. In accordance with the present improved invention there is provided a formulation where cold-pressed, organic safflower oil, flaxseed oil, jojoba oil, sweet almond oil, apricot kernel oil, tocopheral linoleate, essential oil of lavender and tincture of benzoin, can be employed to aid in the non-greasy treatment and alleviation of chronic skin conditions. The embodiment of the improved invention follow to illustrate the formulation.

The main ingredients of the liquid cleansing compostion are:

100 to 5000 milliliters liquid glycerin soap, purchased from S.F.J.C. Corp, 3241 Monument Way, Suite 1, Concord, Calif. 94518,

0.1 to 100 milliliters of a perfuming ingredient such as oil of lavender blossom,

0.1 to 300 milliliters cold-pressed safflower oil,

0.1 to 300 milliliters cold-pressed flaxseed oil,

0.1 to 300 milliliters jojoba oil,

0.1 to 300 milliliters sweet almond oil,

0.1 to 300 millililters apricot kernel oil,

0.1 to 300 milliliters tocopheral linoleate,

0.01 to 100 milliliter tincture of benzoin,

My liquid soap process is as follows:

placing glycerin liquid soap, flaxseed oil, safflower, jojoba, sweet almond oil, apricot kernel oil, tocopheral linoleate, and tincture of benzoin in a partially submerged container over hot water to form a mixture,

heating said mixture until mixture reaches a temperature of 80 degrees C;

removing said mixture from heat, and stirring said mixture until it cools to 35 degrees C;

adding perfuming ingredient such as essential oil of lavender blossom, and dmdm hydantoin to mixture,

and stirring said mixture until mixture cools,

whereby the ingredients are formed into a liquid soap.

It is believed that the combination and high volume of organic cold-pressed flaxseed oil, safflower oil lipids, tocopheral linoleate, and tincture of benzoin, combined in the liquid soap, cleanse, restore and deliver nutrients that are essential for the maintenance of healthy skin, as well as alleviating, and treating conditions attributed to lupus, rosacea, dermatitis and eczema skin conditions but we don't wish to be bound by this.

As noted, the improved composition hereof comprises a topical application which is applied to the dermal areas of the body to alleviate and treat rosacea, lupus, dry, chapped, flaky, rough, reddened, cracked, and chronic dry skin conditions, by supplying to the skin the essential polyunsaturated oil lipids it needs to maintain its healthy condition.

The improved formulation is highly effective for cosmetic, alternative therapy, and pharmaceutical uses in the treatment of lupus, dermatitis, eczema and dry skin due to the resistance of the formulations to attack by body liquids, sebaceous oils and perspiration.

The improved formulation provides a long lasting film on the skin that is applied while washing, showering or bathing.

The improved formulation employs organic polyunsaturated oil lipids in the place of mineral oil, which by its solvent action strips skin surface lipids.

The improved formulation cleanses and hydrates the skin.

The improved formulation does not strip the acid mantle from the skin.

The improved formulation is suitable for infant, adolescent and aged care use.

For a more complete understanding of the present improved composition reference is made to the following examples. The following examples are illustrative of the present improved composition and are not intended in any as a limitation upon the scope thereof.

### EXAMPLE 1

The composition of the liquid soap was applied once daily to the hands of a woman suffering from a dry, flaky skin condition. Within four days of use, the flakiness disappeared.

### EXAMPLE 2

A female user, suffering from red chapped hands, found the liquid soap calmed her skin, and restored it to a healthy condition.

### EXAMPLE 3

To evidence the efficacy of the bar soap composition, a series of individuals suffering from dry skin, were treated with the liquid soap formulation. Compared to commercially available liquid soaps, the results based upon the efficacy of the present invention are set forth hereinafter:

a) adolescent user found the present invention alleviated dry, flaky skin on her arms and legs caused by moisture depletion from the skin due to the effects of warm air-conditioning;

b) female user found the present invention moisturized her skin after shaving her legs;

c) female users found the present invention did not strip the natural oils from their skin compared to commercially available soaps;

d) the present invention was used by a female dermatitis sufferer, who found the skin on her hands did not dry out after repeated hand washing;

f) all users found the present invention to have a deeply hydrating effect that remained on the skin until the next bathing period;

g) all users found the present invention to be superior to commercially available soaps and skin cleansers.

From the preceding it will be readily perceived by the reader that an urgent need exists for this improved composition for the alleviation of chronic skin conditions denoted heretofore, but which also, can be used to prevent such conditions from occurring;

The improved composition provides:

an organic, solvent free formulation;

an organic antiseptic to fight bacterial, and environmental skin aggressors,

a composition that has immediate effectiveness in the treatment of chronic skin infirmities,

superior skin penetration and nutrient supplying properties,

high volumes of organic polyunsaturated oils that replace lost dermal lipids,

fills an urgent need in the pharmaceutical, alternative health, and cosmetic markets,

organic calming, moisturizing compounds that do not strip the acid mantle from skin,

Deep moisturizing effects while cleansing the skin;

a non-greasy moisturizing film on the skin that is resistant to attack by body fluids, sebaceous oils and perspiration;

organic oil lipids in the place of mineral oil, which by its solvent action strips skin surface lipids; and an

organic low sudsing composition that leaves no drying residue on skin.

While the above description contains many specificity's, these should not be construed as limitations on the scope of the invention, but merely providing illustrations of some of the presently preferred embodiments of this invention.

Many other variations are possible. For example, it is additionally possible to add to the present invention many other adjuvants such as color pigments, pearling pigments, additional essential oils and perfuming ingredients, oatmeal, almond meal, vitamins, antibiotics, anti-inflammatory, menthol, camphor, and lathering additives, floral waters, milk, honey, vegetable, fruit and plant juices, dried vegetable and fruit peels, alpha-hydroxy, glycolic, lactic, salicylic, exfoliating fruit acids, vegetable, nut, and plant oils and still be within the purview of the invention.

Accordingly, the scope of the invention should be determined not only by the embodiments illustrated, but by the appended claims and their legal equivalents, rather than by the examples given.

What is claimed is:

1. A liquid soap comprising:
from 10 to 5000 milliliters liquid glycerin soap,
from 0.2 to 300 milliliters sweet almond oil,
from 0.1 to 300 milliliters safflower oil,
from 0.1 to 300 milliliters apricot kernel oil,
from 0.1 to 300 milliliters flaxseed oil,
from 0.1 to 100 milliliters tincture of benzoin,
from 0.1 to 300 milliliters tocopheral linoleate,
from 0.1 to 200 milliliters jojoba oil, and
from 0.1 to 100 milliliters of perfuming ingredient such as essential oil of lavender.

2. A liquid soap comprising:
500 milliliters liquid glycerin soap,
5 milliliters essential oil of lavender blossom,
5 milliliters cold-pressed safflower oil,
2.5 milliliters cold-pressed flaxseed oil,
2.5 milliliters jojoba oil,
2.5 milliliters sweet almond oil,
2.5 millililters apricot kernel oil,
2.5 milliliters tocopheral linoleate, and
2.5 milliliter tincture of benzoin.

3. A liquid soap as in claim 2 further comprising 0.6 milliliters of a preservative such as dmdm hydantoin.

4. A method of making liquid soap comprising:
placing glycerin liquid soap, flaxseed oil, safflower, jojoba, sweet almond oil, apricot kernel oil, tocopheral linoleate, and tincture of benzoin in a partially submerged container over hot water to form a mixture,
heating said mixture until mixture reaches a temperature of 80 degrees C.,
removing said mixture from heat, and stirring said mixture until it cools to 35 degrees C,
adding a perfuming ingredient such as essential oil of lavender blossom, and
a preservative such as dmdm hydantoin to said mixture, and
stirring said mixture until mixture cools, whereby the ingredients are formed into a liquid soap.
